(19) United States
(12) Patent Application Publication
(10) Pub. No.: US 2011/0028789 A1
(43) Pub. Date: Feb. 3, 2011

(54) SURGICAL ACCESS APPARATUS AND METHODS

(76) Inventor: Jeffery Thramann, Longmont, CO (US)

Correspondence Address: Holland & Hart, LLP (LANX, Inc.);Intellectual Property Department, P.O. Box 8749, Denver, CO, 80201-8749, US

(21) Appl. No.: 12/680,723

(22) PCT Filed: Oct. 15, 2008
(86) PCT No.: PCT/US08/80014

§ 371 Date: Sep. 1, 2010

(30) Foreign Application Priority Data
Oct. 15, 2007 (US) 60980020

(51) Int. Cl. A61B 1/317; A61B 17/56

(52) U.S. Cl. 600/183

(57) ABSTRACT

There is disclosed a method of gaining access to a lateral side of a spinal column for a spinal fusion procedure at a surgical site. In an embodiment, the method includes inserting a distal end of a shaft through skin at an entry point into a patient; steering the shaft toward a surgical site using a visualization device to avoid damage to structures between the entry point and the surgical site; and retracting tissue between the entry point and the surgical site to define a pathway for access to the lateral side of the spinal column for a spinal fusion procedure. Other embodiments are also disclosed.

### REFERENCE TO PENDING PRIOR PATENT APPLICATION

This application claims the benefit of U.S. Provisional Patent Application No. 60/980,020, filed Oct. 15, 2007 by Jeffrey Thramann for SURGICAL ACCESS APPARATUS AND METHODS, which patent application is hereby incorporated herein by reference.

### FIELD OF THE INVENTION

The invention relates to devices and methods for minimally invasively accessing a surgical site.

### BACKGROUND

A variety of surgical interventions require access to sites within a patient's body. Traditionally, open approaches have been used to gain access to the surgical site. In an open approach, relatively large incisions are made and the skin, fat, muscle, and other tissues are retracted to allow direct visualization and direct treatment by placement of the surgeon's hands and instruments into the incision. While open approaches provide very good visualization and convenience to the surgeon, they may result in much disruption of the tissues surrounding the surgical site and may require lengthy recovery periods for the tissues to heal. They may also result in weakening of muscles, tendons, ligaments, and other tissues with long term reduction in function.

Over the past several years, more and more surgical treatments have incorporated less invasive, or minimally invasive, approaches to reduce trauma and speed recovery. For example, treatment of the soft tissues of the knee and shoulder has transitioned almost entirely from open approaches to minimally invasive approaches using small diameter arthroscopes inserted into the joint space for visualization and surgical instruments inserted through puncture wounds or tubes into the joint space. Similarly, endoscopic surgery for treating the abdomen has become prevalent. Interventions such as appendectomy, gall stone removal, treatment of endometriosis, gastric bypass, and many other procedures are now performed through small incisions.

In more recent years, procedures have been developed to implant various devices into the body using minimally invasive techniques. Implants such as hip joint replacements, knee joint replacements, and intervertebral spacers have been placed through small incisions with visualization through a scope, X-ray visualization, triangulated navigation, or by using surgical landmarks.

For example, a variety of treatments for spine disease involve surgical approaches to the spinal column. Posterior access to the spinal column has been used, e.g., for discectomy, interbody fusion, pedicle screw stabilization, posterior plate spinal stabilization, debridement, and other procedures. Anterior access to the spinal column has been used, e.g., for interbody fusion and anterior plate spinal stabilization. Lateral access to the spinal column has been used, e.g., for discectomy and interbody fusion. All of these approaches have been proposed both as open procedures and minimally invasive procedures. Each procedure has its particular advantages and disadvantages in terms of difficulty in gaining access to the surgical site and risk to nearby structures such as nerves and vessels. For example, in a posterior approach, the bony structure of the vertebrae can be difficult to work around and the spinal cord and major nerve roots may be injured. In an anterior approach, the surgeon must penetrate deep into the abdomen and organs, the bowel, and major vessels may be injured. Similarly, in a lateral approach, the surgeon must penetrate deep into the abdomen and organ, the bowel, major vessels, and nerves may be injured.

Careful preoperative planning and intraoperative X-ray are helpful in avoiding injury to nearby structures. Electrical stimulation with nerve monitoring has been utilized to warn of proximity to nerves. However, the interpretation of these tools can be ambiguous and reliability has been questioned. This is especially true in the case of electrical stimulation and nerve monitoring which require a very highly skilled technician monitoring several nerve pathways to be successful.

### SUMMARY OF THE INVENTION

In an embodiment, there is provided a method of gaining access to a lateral side of a spinal column for a spinal fusion procedure at a surgical site, the method comprising inserting a distal end of a shaft through skin at an entry point into a patient; steering the shaft toward a surgical site using a visualization device to avoid damage to structures between the entry point and the surgical site; and retracting tissue between the entry point and the surgical site to define a pathway for access to the lateral side of the spinal column for a spinal fusion procedure.

In another embodiment, there is provided a method of gaining access to a surgical site, the method comprising inserting a distal end of a shaft through skin at an entry point into a patient; steering the shaft toward the surgical site using a visualization device to avoid damage to structures between the entry point and the surgical site; and retracting tissue between the entry point and the surgical site to define a pathway to the surgical site.

Other embodiments are also disclosed.

### BRIEF DESCRIPTION OF THE DRAWINGS

Various examples of the present invention will be discussed with reference to the appended drawings. These drawings depict only illustrative examples of the invention and are not to be considered limiting of its scope.

FIG. 1 is a cross sectional view of a human abdomen at the level of the lumbar spine;

FIG. 2 is an elevation view of a surgical access apparatus according to the present invention;

FIG. 3 is an elevation view of a surgical access apparatus like that of FIG. 2 but with a flexible shaft;

FIG. 4 is a cross sectional view taken along lines 4-4 of FIG. 3;

FIG. 5 is a is a cross sectional view taken along lines 5-5 of FIG. 4;

FIG. 6 is a is a cross sectional view taken along lines 6-6 of FIG. 4;

FIG. 7 is a cross sectional view of a human abdomen with the apparatus of FIG. 2 in use to define a path to a surgical site;

FIG. 8 is an elevation view of a monitor displaying the view from a camera in the apparatus of FIG. 2;

FIG. 9 is a cross sectional view of a human abdomen with the apparatus of FIG. 2 in use to define a path to a surgical site;

FIG. 10 is a cross sectional view like that of FIG. 4 showing a sleeve around the apparatus of FIG. 2;

FIG. 11 is an elevation view of a set of tubes for use with the apparatus of FIG. 2;

FIG. 12 is a cross sectional view of a human abdomen with a tube of FIG. 11 in place; and

FIG. 13 is a view through the tube of FIG. 12.

### DESCRIPTION OF THE ILLUSTRATIVE EXAMPLES

The surgical access apparatus and methods of the present invention may be utilized to gain access to any number of surgical sites. Such sites may include the hip joint, knee joint, shoulder joint, elbow joint, ankle joint, digital joint of the hand or foot, fracture site, tumor site, vertebral body, disc space, pellicles, facet joints, spinal canal, spinal processes, and/or other surgical sites. The apparatus and methods may be utilized to approach the site from a variety of directions including anteriorly, posteriorly, laterally, obliquely, and/or other directions.

The apparatus utilizes a device for visualizing a path created from the outside of the patient to the surgical site. The visualization device may include, e.g., an endoscope, fluoroscope, X-ray, and/or surgical navigation system. For example, an endoscope (referred to as a scope throughout the rest of this specification) may be used to dissect a path through body tissues to the surgical site. The scope may be pressed through the tissues to separate the tissues along planes, e.g., along fascial planes between muscle tissue, organs, vessels, nerves, and other tissues. The scope may have additional features such as an inflatable tip, water jet, lubricant, and/or other features to ease its passage through the tissues. As the scope is advanced, the surgeon may view the surrounding tissue and steer the scope away from nerves, vessels, and other structures that it is desirable to avoid. The surgeon may also steer the scope along natural separation planes, through muscles, and/or along any other desirable path.

The scope may be rigid such that it transcribes a straight path through the tissues. The surgeon may manipulate such a rigid scope to press tissues one way or the other and work the scope along a desired path. The scope may be flexible so that it can snake around structures and follow a non-linear path through the tissues. The scope may be steerable so that the angle of the tip and/or curvature of the scope shaft can be controlled by the user to more precisely direct the shaft along a desired path. A flexible scope may be constructed so that it can be straightened once it has defined a path to the surgical site to straighten the path and ease access to the site. For example, the scope body may receive a rigid obturator and/or overlying tube to straighten the path and guide the obturator and/or tube to the surgical site. The scope body may be pressurized with a fluid to straighten the scope body. For example, fluids such as CO₂ and/or other gasses and saline and/or other liquids may be used to pressurize and straighten the scope body. The scope may be straightened by tensioning cables or other members. For example a steerable scope may have actuation members that can be used to bend the scope and subsequently straighten it.

The scope may be used to place a sleeve, cable, wire, and/or other elongated member at the surgical site which is subsequently used to straighten the path to the site. For example, the scope may be placed in a sleeve prior to guiding the scope to the surgical site. Once the site has been reached, the scope may be withdrawn leaving the sleeve in place. The scope may carry a wire or cable that flexes with the scope and which is left in place when the scope is retracted. Alternatively, a wire, cable, rod, tube, and/or other elongated member may be guided to the surgical site using a fluoroscope, X-ray, surgical navigation system, and/or other visualization device. Preferably, the elongated member is steerable to follow a non-linear path through the tissues. An obturator, fluid pressure, tube, and/or other device may then be used to straighten the elongated member and therefore the path to the site. Dilators, retractors, tubes, and/or other tissue opening and/or holding devices may be guided with the scope, sleeve, wire, obturator or otherwise to the site to create a tunnel through which the surgical procedure may be performed.

The following illustrative examples illustrate the apparatus and methods of the present invention in use to access the lateral side of the spinal column for a spinal fusion procedure. However, the examples are illustrative only and the apparatus and methods may be used at any surgical site in any surgical approach where it is desirable to safely develop a pathway to the surgical site.

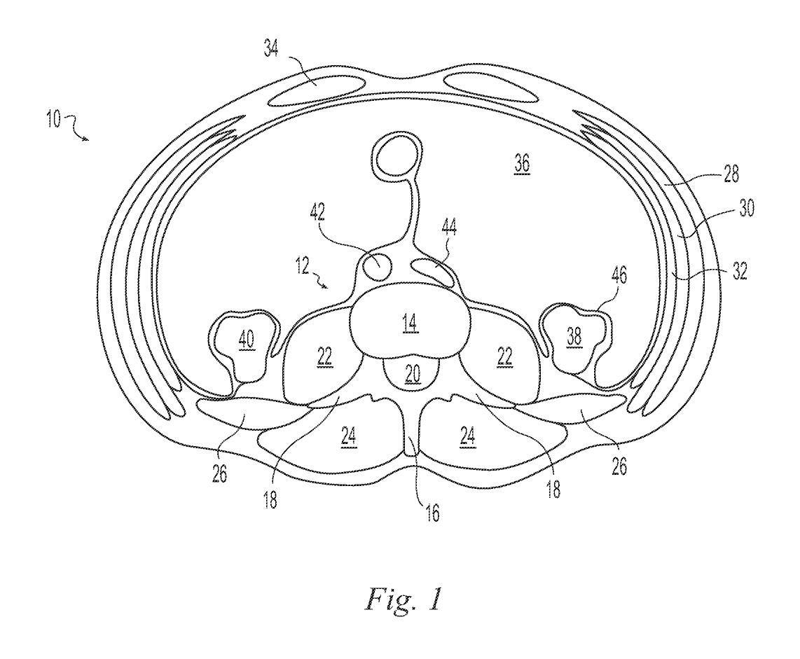

FIG. 1 illustrates a cross section of a human abdomen 10 at the level of the lumbar spine. The vertebra 12 is near the center of the abdomen 10 and includes a vertebral body 14, a posteriorly projecting spinous process 16, and transverse processes 18 defining the spinal canal 20. The psoas major muscles 22 lie laterally on each side of the vertebra 12 and the erector spinae muscles 24 lie posteriorly on either side of the spinous process 16. The quadratus lumborum muscles 26 are shown between the psoas major 22 and erector spinae 24 at the terminus of the transverse processes 18 and extending laterally outwardly. The abdominal wall includes the external oblique, internal oblique, and transverse abdominal muscles 28, 30, 32 laterally and the rectus muscles 34 anteriorly. The abdominal cavity 36 contains the ascending and descending colon 38, 40 laterally; the aorta 42 and inferior vena cava 44 anteriorly; and the peritoneum 46. The nerves that populate this region are not shown. Of particular interest is the lumbar plexus, including the genitofemoral nerve, which passes through the psoas major muscle 22. The apparatus and method of the present invention will be demonstrated in used to safely create a surgical path from the lateral side of the abdomen 10 through the intervening tissues to the lateral side of the vertebral body 14.

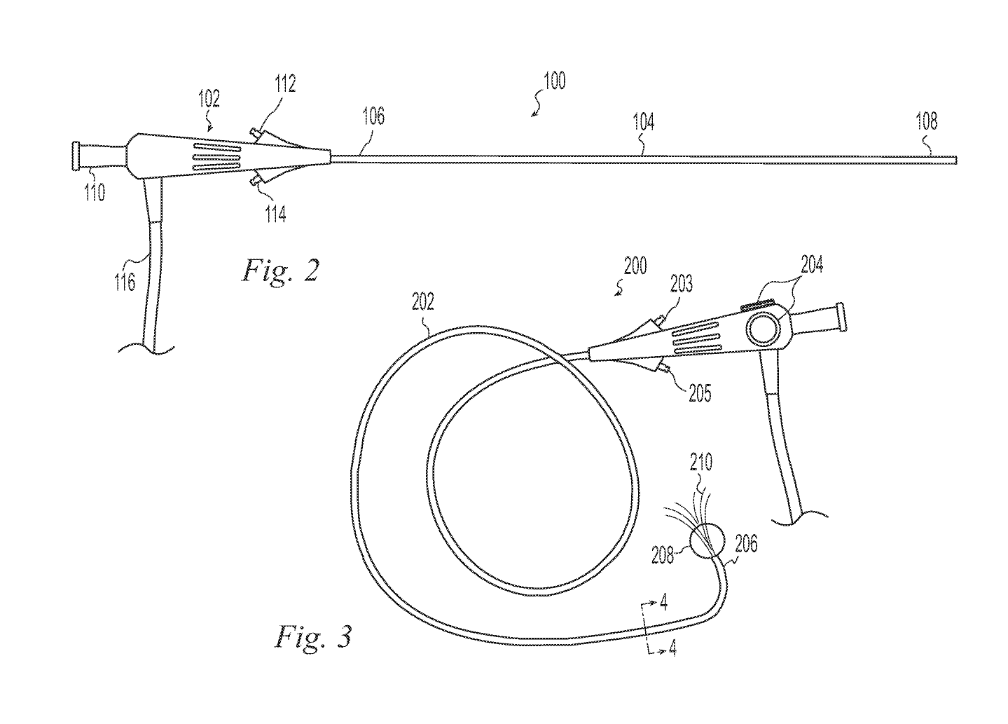

FIG. 2 illustrates a rigid endoscope 100 having a body 102 and a shaft 104. The shaft 104 includes a proximal end 106 and a distal end 108. The proximal end 106 is attached to the body 102. The body 102 includes an observation end 110, a first port 112, a second port 114 and a cable 116. The endoscope 100 includes a viewing system able to transmit an image from the distal end 108 to the observation end 110. The observation end 110 may include a lens for direct viewing or a connection for transmitting the image to a remote monitor. With the use of miniaturized cameras, a camera may be placed at the distal end 108 and the image carried back to the viewing end or remote monitor by an electrical conductor. The ports 112, 114 conduct fluids to the shaft 104. The cable 116 provides power to the scope 100, and may provide light for illuminating the surgical site.

FIG. 3 illustrates a flexible, steerable endoscope 200 similar to that of FIG. 2 including a shaft 202 and ports 203, 205. However, the shaft 202 can be curved to follow a non-linear path through the abdomen 10 by operating actuators 204. The shaft 202 includes a distal end 206 having a balloon 208 that may be selectively inflated (as shown in FIG. 3) to separate tissues at the distal end and then deflated to ease advancement of the shaft into the tissues. Alternatively, or in combination with a balloon 208, a fluid jet 210 may be provided that may be selectively activated to separate tissues and or lubricate the path of the shaft 202.

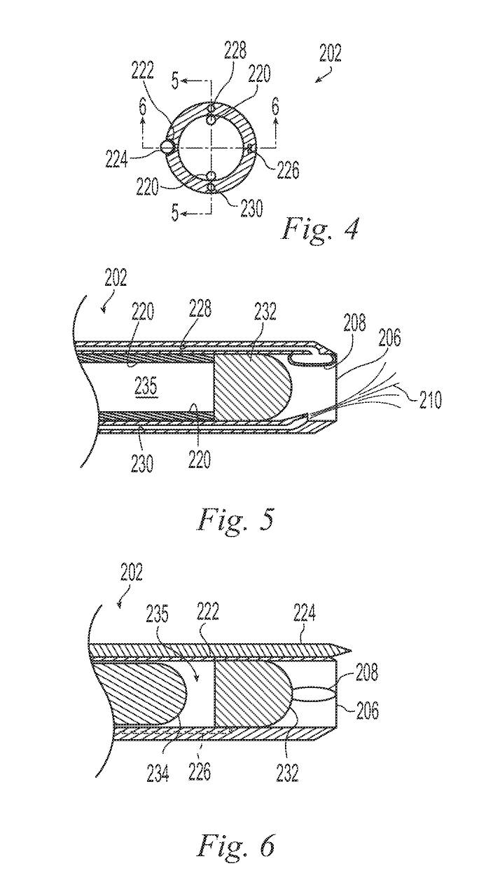

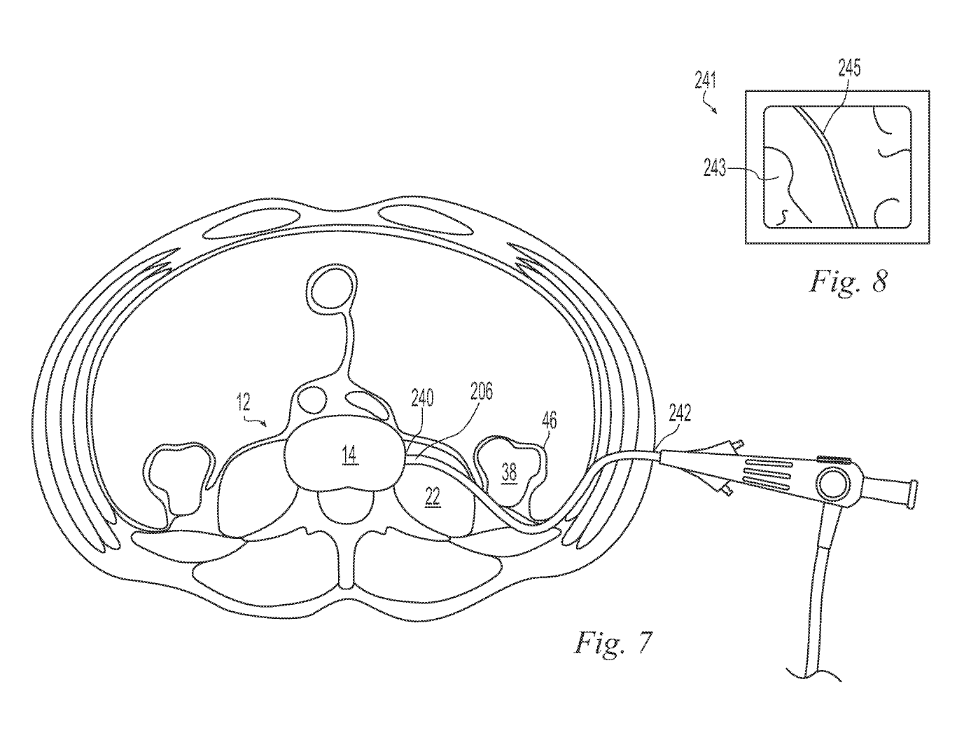

FIG. 4 is a cross sectional view of the shaft 202 of the flexible, endoscope 200 taken along lines 4-4 of FIG. 3, FIG. 5 is a cross sectional view of the shaft 202 taken along lines 5-5 of FIG. 4, and FIG. 6 is a cross sectional view of the shaft 202 taken along lines 6-6 of FIG. 4. The rigid endoscope 100 of FIG. 2 may also have a similar cross-sectional geometry. Cables 220 are connected to the actuators 204 to steer the shaft 202. A groove 222 formed in the sidewall of the shaft 202 receives a wire, cable, obturator, and/or other elongated member 224 to be carried or guided by the shaft 202 and/or to be used to straighten the shaft 202. For example, the groove 222 may carry a K-wire to the surgical site as the scope is advanced or a K-wire may be inserted into the groove 222 to guide it to the surgical site after the scope has been advanced to the surgical site. Conductors 226 are embedded in the shaft 202 to carry images from a camera at the tip of the shaft to a monitor 241 (FIG. 8). Fluid conduit 228 communicates with port 203 and carries fluid for expanding the balloon 208 and fluid conduit 230 communicates with port, 205 and carries fluid for producing the fluid jet 210. The balloon 208 is shown deflated in FIGS. 5 and 6. Upon inflation through conduit 228, the balloon expands out of the hollow distal end 206 of the shaft 202. A camera 232 is mounted near the distal end 206 and is connected to conductors 226 to transmit images to the monitor 241. An obturator 234 is insertable into the shaft 202 to straighten the shaft 202. Alternatively, the cables 220 may be actuable to straighten the shaft 202. In another embodiment, fluid is pressurized in the hollow interior 235 of the shaft 202 to straighten the shaft 202.

FIG. 7 illustrates the flexible endoscope 200 in use to access a surgical site 240 on the lateral side of the vertebra 12. The distal end 206 of the shaft 202 is inserted into a stab incision 242 through the skin, oblique muscles, and transverse muscle. In the illustrative example of FIG. 7, the shaft 202 is then steered posteriorly behind the peritoneum 46, behind the ascending colon 38, and into the psoas major muscle 22. Visualization provided by the camera 232 allows the surgeon to carefully direct the path of the shaft 202 and avoid damage to the peritoneum, colon, and other structures. As the distal end 206 of the shaft is advanced through the psoas major muscle 22, visualization allows the surgeon to avoid impinging the nerves of the lumbar plexus and in particular the genitofemoral nerve. For example, an image may be displayed on a monitor as shown in FIG. 8. In the illustrative example of FIG. 8, the surgeon can see the tissues 243 of the psoas major muscle and a nerve 245, such as the genitofemoral nerve, passing through the tissues 243. Since the nerve 245 is in the path of the scope 200, the surgeon manipulates the distal end 206 of the shaft 202 to avoid the nerve 245 and continues to advance the shaft 202. Once the distal end 206 reaches the surgical site 240, the scope can be used to assess the condition of the surgical site 240. Fluoroscopy or other visualization techniques may be used in combination with the scope 200 to precisely locate the path to the surgical site 240.

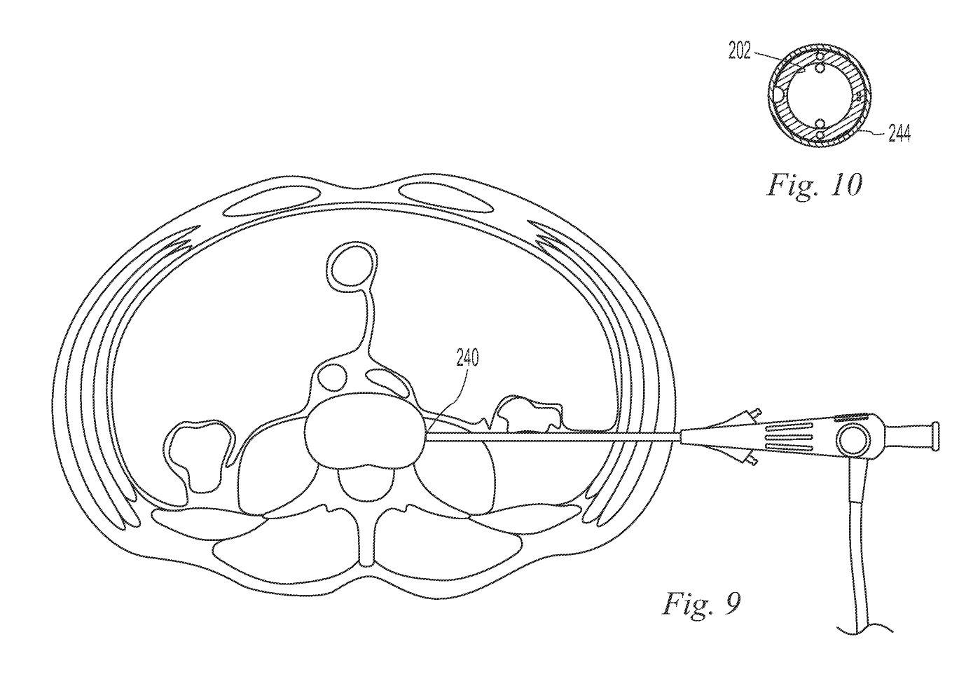

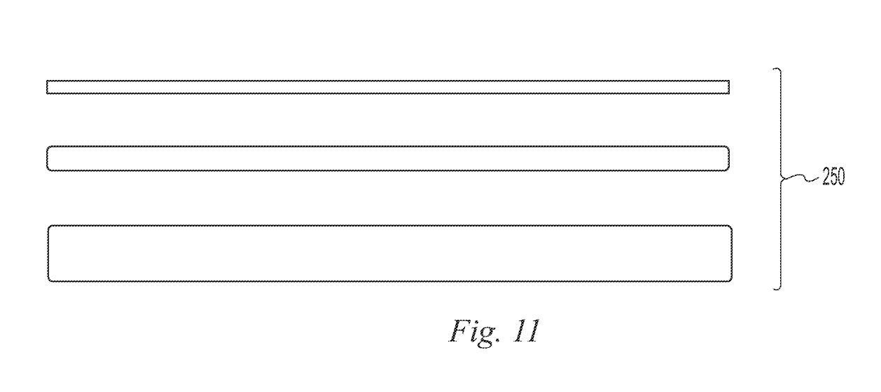

The shaft 202 is now straightened to the configuration shown in FIG. 9. Straightening the shaft 202 safely retracts the tissues. In the illustrative example of FIG. 9, the shaft is straightened by sliding the obturator 234 into the shaft 202. Once it is straightened, the K-wire 224 is inserted along the groove 222 and driven into the surgical site 240 to preserve the path to the surgical site 240 when the scope 200 is withdrawn. It is also contemplated that the K-wire 224 itself may be sufficiently rigid to straighten the shaft 202 as it is inserted along the groove 222 and that the obturator 234 may be omitted. Alternatively, fluid may be pressurized in the shaft 202 and/or the cables 220 may be actuated to straighten the shaft 202. In yet another alternative, a sleeve 244, may be carried by the shaft 202, as shown in FIG. 10, and left in place as the shaft 202 is withdrawn. The sleeve 244 may then be straightened using any of the previously described methods or other suitable methods.

The rigid scope 100 of FIG. 2 may be used to define the path to the surgical site 240 rather than the flexible scope 200 of FIG. 3. The distal end 108 is manipulated to avoid impinging nerves, vessels and other structures that need to be avoided with the aid of visualization provided by the camera 232. When the distal end 108 reaches the surgical site 240, the path is already straight due to the rigid nature of the shaft 104.

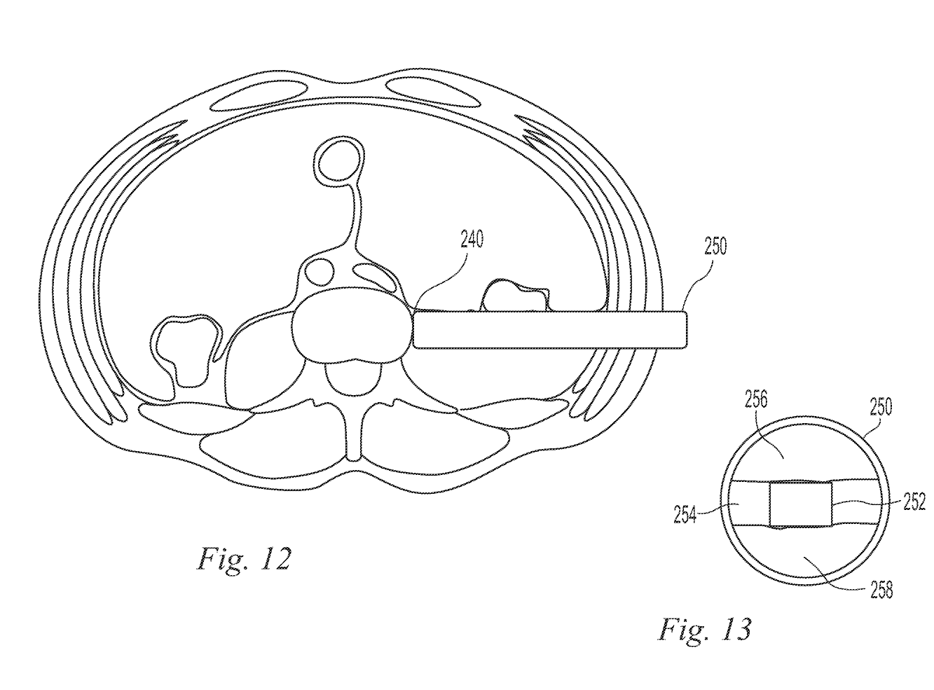

Once a path has been established to the surgical site 240, any desired surgical procedure may be carried out. FIG. 11 illustrates a set of nesting tubes 250. The tubes 250 may be sequentially placed along the surgical path, such as over the K-wire 224 to gently dilate the path. With a large tube 250 in place as shown in FIG. 12, a surgeon can access the surgical site 240 to carry out a procedure. FIG. 13 illustrates a view down the tube 250 showing an exemplary procedure in which an intervertebral spacer 252 is placed into a disc space 254 between two vertebral bodies 256, 258.

Although examples of a surgical access apparatus and its use have been described and illustrated in detail, it is to be understood that the same is intended by way of illustration and example only and is not to be taken by way of limitation. The invention has been illustrated as an endoscope used to define a path to a surgical site on the lateral side of a vertebral body to implant an intervertebral spacer. However, the surgical access apparatus may be configured for use at other locations within a patient's body to access other surgical sites to perform other surgical procedures. The apparatus may use visualization techniques other than an endoscope to guide it to the surgical site. Accordingly, variations in and modifications to the surgical access apparatus and its use will be apparent to those of ordinary skill in the art and still fall within the scope of the invention.

What is claimed is:

1. A method of gaining access to a lateral side of a spinal column for a spinal fusion procedure at a surgical site, the method comprising:
inserting a distal end of a shaft through skin at an entry point into a patient;
steering the shaft toward a surgical site using a visualization device to avoid damage to structures between the entry point and the surgical site; and
retracting tissue between the entry point and the surgical site to define a pathway for access to the lateral side of the spinal column for a spinal fusion procedure.

2. A method in accordance with claim 1, wherein inserting the distal end of the shaft through the skin at the entry point into the patient includes inserting the distal end of the shaft through a stab incision through the skin.

3. A method in accordance with claim 1, wherein inserting the distal end of the shaft through the skin further includes inserting the distal end of the shaft through oblique muscles and transverse muscles.

4. A method in accordance with claim 1, wherein steering the shaft includes directing the distal end of the shaft behind peritoneum, behind an ascending colon, and into a psoas major muscle.

5. A method in accordance with claim 4, further comprising observing nerves, and avoiding contact with the nerves, as the distal end of the shaft if directed into the psoas major muscle.

6. A method in accordance with claim 5, further comprising assessing the surgical site with the visualization device.

7. A method in accordance with claim 1, further comprising observing nerves, and avoiding contact with the nerves, as the distal end of the shaft is directed between the entry point and the surgical site.

8. A method in accordance with claim 1, further comprising assessing the surgical site with the visualization device.

9. A method in accordance with claim 1, wherein the visualization device includes at least one of an endoscope, a fluoroscope, an X-ray system, and a surgical navigation system.

10. A method in accordance with claim 1, wherein steering the shaft toward the surgical site is from a lateral direction.

11. A method in accordance with claim 1, further comprising dissecting a path through the tissues to the surgical site by pressing the distal end of the shaft through the tissues to separate the tissues along planes.

12. A method in accordance with claim 11, further comprising using at least one of an inflatable balloon tip, a fluid jet, and lubricant to aid passage of the distal end of the shaft through the tissues.

13. A method in accordance with claim 1, wherein steering the shaft toward the surgical site includes steering the distal end of the shaft along natural separation places and through muscles.

14. A method in accordance with claim 1, wherein retracting the tissue between the entry point and the surgical site includes inserting and steering the shaft in a rigid configuration to the surgical site, and further includes moving a portion of the shaft proximal of the distal end so as to retract the tissue.

15. A method in accordance with claim 1, wherein retracting the tissue between the entry point and the surgical site includes inserting and steering the shaft in a flexible configuration to the surgical site, and further includes adjusting the shaft from the flexible configuration to a rigid configuration, and moving a portion of the shaft proximal of the distal end so as to retract the tissue.

16. A method in accordance with claim 15, further comprising straightening the shaft with one or an obturator, a tube overlying the shaft, pressurizing fluid into the shaft, tensioning cables, and actuation members for selectively bending and straightening the shaft.

17. A method in accordance with claim 1, further comprising placing of at least one of a sleeve, a cable, a wire, and an elongated member at the surgical site, and straightening the pathway to the surgical site with the at least one of the sleeve, the cable, the wire, and the elongated member subsequent to the placing thereof.

18. A method in accordance with claim 1, further comprising guiding at least one of a dilator, a set of nesting tubes, a retractor, a tissue opening device, and a tissue holding device to the surgical site with the shaft.

19. A method in accordance with claim 18, further comprising removing the shaft from the surgical site, and preserving the pathway with the at least one of the dilator, the retractor, the tissue opening device, and the tissue holding device remaining in the surgical site.

20. A method in accordance with claim 1, further comprising guiding a K-wire through the shaft to the surgical site.

21. A method in accordance with claim 20, further comprising removing the shaft from the surgical site, and preserving the pathway with the K-wire remaining in the surgical site.

22. A method of gaining access to a surgical site, the method comprising:
inserting a distal end of a shaft through skin at an entry point into a patient;
steering the shaft toward the surgical site using a visualization device to avoid damage to structures between the entry point and the surgical site; and
retracting tissue between the entry point and the surgical site to define a pathway to the surgical site.

23. A method in accordance with claim 22, wherein the surgical site is one chosen from one of a hip joint, an elbow joint, an ankle joint, a digital joint of a hand, a digital joint of a foot, a fracture site, a tumor site, a vertebral body, a disc space, pedicles, facet joints, spinal canal, and spinal processes.

24. A method in accordance with claim 22, wherein steering the shaft toward the surgical site is from at least one of an anterior direction, a posterior direction, a lateral direction, and an oblique direction.

25. A method in accordance with claim 22, wherein the visualization device includes at least one of an endoscope, a fluoroscope, an X-ray system, and a surgical navigation system.

26. A method in accordance with claim 22, further comprising dissecting a path through the tissues to the surgical site by pressing the distal end of the shaft through the tissues to separate the tissues along planes.

27. A method in accordance with claim 26, further comprising using at least one of an inflatable balloon tip, a fluid jet, and lubricant to aid passage of the distal end of the shaft through the tissues.

28. A method in accordance with claim 22, wherein steering the shaft toward the surgical site includes steering the distal end of the shaft along natural separation places and through muscles.

29. A method in accordance with claim 22, further comprising straightening the shaft with one or an obturator, a tube overlying the shaft, pressurizing fluid into the shaft, tensioning cables, and actuation members for selectively bending and straightening the shaft.

30. A method in accordance with claim 2, further comprising placing of at least one of a sleeve, a cable, a wire, and an elongated member at the surgical site, and straightening the pathway to the surgical site with the at least one of the sleeve, the cable, the wire, and the elongated member subsequent to the placing thereof.

31. A method in accordance with claim 22, further comprising guiding at least one of a dilator, a set of nesting tubes, a retractor, a tissue opening device, and a tissue holding device to the surgical site with the shaft.

32. A method in accordance with claim 31, further comprising removing the shaft from the surgical site, and preserving the pathway with the at least one of the dilator, the retractor, the tissue opening device, and the tissue holding device remaining in the surgical site.

33. A method in accordance with claim 22, further comprising guiding a K-wire through the shaft to the surgical site.

34. A method in accordance with claim 33, further comprising removing the shaft from the surgical site, and preserving the pathway with the K-wire remaining in the surgical site.

35. Apparatus for gaining access to a lateral side of a spinal column for a spinal fusion procedure at a surgical site, the apparatus comprising:
a shaft having a distal end configured for insertion though skin at an entry point into a patient;
a visualization device in attachments to the shaft, the visualization device being configured for steering the shaft toward a surgical site so as to avoid damage to structures between the entry point and the surgical site; and
the shaft being configured for retracting tissue between the entry point and the surgical site to define a pathway for access to the lateral side of the spinal column for a spinal fusion procedure.
